(19) United States
(12) Plant Patent Application Publication
(10) Pub. No.: US 2003/0121084 P1
(43) Pub. Date: Jun. 26, 2003

(54) Apple tree named 'Nicoter'

(76) Inventor: Johan Nicolai, Sint-Truiden-Gorsem (BE)

Correspondence Address: STRATTON BALLEW, 213 S 12TH AVE, YAKIMA, WA, 98902, US

(21) Appl. No.: 10/293,207

(22) Filed: Nov. 12, 2002

Related U.S. Application Data
(60) Provisional application No. 60/337,600, filed on Nov. 13, 2001.

(51) Int. Cl. A01H 5/00

(52) U.S. Cl. PLT/161

(57) ABSTRACT

A new apple tree is disclosed. The new variety is the result of a cross between ‘Gala’ and ‘Braeburn,’ and is most similar in appearance to ‘Gala.’ The new variety is distinguishable from ‘Gala’ by its red coloration and slightly less conical shape.

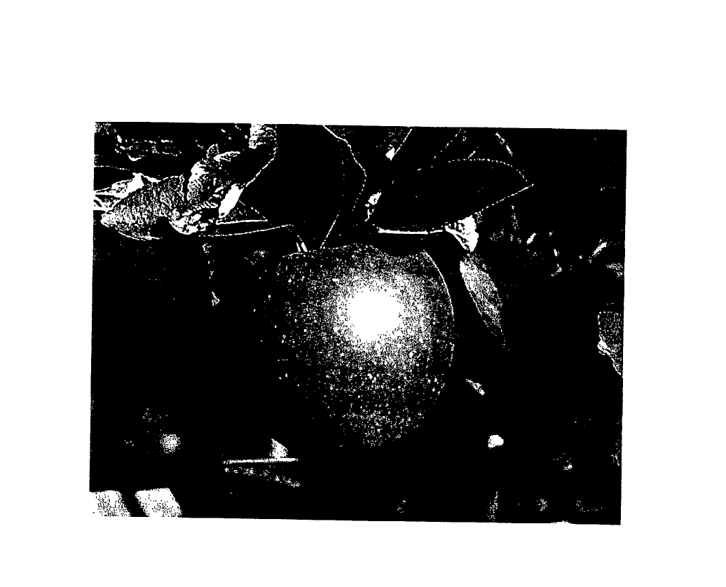

### PRIORITY CLAIM

This application claims priority under U.S. Provisional Application No. 60/337,600 filed Nov. 13, 2002.

### FIELD OF THE INVENTION

The invention relates to the field of fruit trees. More specifically, the invention consists of a new and distinct variety of apple tree (Maluis domestica).

### ORIGIN OF THE VARIETY

The new variety originated as a controlled cross between ‘Gala’ and ‘Braeburn’.

### ASEXUAL REPRODUCTION OF THE VARIETY

The new variety has been asexually propagated, and has been demonstrated to remain true to type through successive generations.

### BRIEF DESCRIPTION OF THE DRAWINGS

The two figures illustrate representative fruit of the new variety.

### DETAILED DESCRIPTION OF THE VARIETY

The following is a detailed botanical description of the new and distinct variety of apple tree, based on observations of specimens grown in Belgium.

The new variety most closely resembles ‘Gala’. However, fruit of the new variety is less conical in shape than ‘Gala.’ The coloration of the new variety is more red and less pink than ‘Gala.’

Tree: Vigor Medium (compare to ‘Golden Delicious’)

Fruit: Shape Short globose conical (compare to ‘Fortune’)

Over color of skin.—Red (compare to ‘Jonathan’).

Time of beginning of flowering (10% open flowers): Medium (compare to ‘Cox's Orange Pippin’)

Time of fruit ripening for eating: Late (compare to ‘Golden Delicious’)

I claim:

1. A new and distinct variety of apple tree as described and claimed herein.
